(19) United States
(12) Patent Application Publication
(10) Pub. No.: US 2023/0365960 A1
(43) Pub. Date: Nov. 16, 2023

(54) Methods for Lysis of Cells Within a Sample

(71) Applicants: MicrobeDX, Inc., Pacific Palisades, CA (US); The Regents of the University of California, Oakland, CA (US)

(72) Inventors: Gabriel MONTI, Cypress, CA (US); Colin Wynn HALFORD, Los Angeles, CA (US); Alexandra PEREBIKOVSKY, Irvine, CA (US); Yujia LIU, Irvine, CA (US); Horacio KIDO, Irvine, CA (US); David Arnold HAAKE, Culver City, CA (US); Marc MADOU, Irvine, CA (US); Daniel GUSSIN, Encinitas, CA (US); Bernard CHURCHILL, Los Angeles, CA (US)

(21) Appl. No.: 18/224,418

(22) Filed: Jul. 20, 2023

Related U.S. Application Data
(63) Continuation of application No. 16/636,259, filed on Feb. 3, 2020.

(51) Int. Cl. C12N 15/10; C12N 9/36; C12N 9/64

(52) U.S. Cl. CPC C12N 15/1003; C12N 9/2462; C12N 9/6489; C12N 2500/12; C12Y 302/01017; C12Y 304/24075

(57) ABSTRACT

There is described a method for extracting a target chemical compound from a cellular material in a sample. The method comprising the steps of: subjecting the sample to mechanical lysis to cause disruption of a cellular membrane in the cellular material; contacting the sample with an alkaline material to produce a lysate composition comprising the target chemical compound; and recovering the lysate composition from the sample. There is also described a method for producing a lysate composition comprising RNA from a mammalian bodily fluid sample comprising a cellular material. There is also described a method for extracting a nucleic acid from a cellular material in a bodily fluid or an inoculant derived therefrom.

### RELATED APPLICATIONS

This application is a continuation of U.S. application Ser. No. 16/636,259, filed on Feb. 3, 2020, which is a 371 of International Patent Application No. PCT/US2018/045211, filed Aug. 3, 2018, which claims the benefit under 35 U.S.C. § 119(e) of provisional patent application No. 62/541,418, filed Aug. 4, 2017, the contents of which are hereby incorporated by reference.

### FIELD OF THE INVENTION

In one of its aspects, the present invention relates to a method for extracting a target chemical compound from a cellular material in a sample. In another of its aspects, the present invention relates to a method for producing a lysate composition comprising RNA from a mammalian bodily fluid sample comprising a cellular material. In yet another of its aspects, the present invention relates to a method for extracting a nucleic acid from a cellular material in a bodily fluid or an inoculant derived therefrom.

### BACKGROUND

The analysis of biological fluid samples, particularly the detection of certain target molecules within a biological fluid has many clinical applications. For example, the isolation and identification of uropathogens in urine samples is an important aspect of the clinical management of patients with urinary tract infections (UTIs) and other infectious diseases.

Culture-based methods for isolating and identifying uropathogens are known in the art, however these methods can be time consuming, labor intensive, and are not cost effective. Recent advances in technology however have allowed for the development of electrochemical DNA biosensors with molecular diagnostic capabilities, including bacterial pathogen detection. In order to run a successful electrochemical assay, a target cell must first be lysed such that RNA is released from within the cell. Thus, the use of electrochemical DNA biosensors relies on the efficient lysis and release of target molecules from the cells to be diagnosed. These cells may include, among others, prokaryotic cells such as Gram-negative bacteria or Gram-positive bacteria, or fungal cells, such as yeast.

There are many conventional lysing techniques that are known to effectively lyse Gram-negative bacteria. For example, chemical lysis using an alkaline solution has been shown to effectively release target molecules, such as 16S rRNA from Gram-negative cells. Because of the thicker cell walls associated with Gram-positive organisms however, this technique is not capable of lysing Gram-positive cells sufficiently for electrochemical detection.

Attempts have been made to develop universal lysing techniques that can effectively release target molecules from a variety of cells including Gram-negative organism, Gram-positive organisms and eukaryotic organisms such as yeast. The only lysis method to date that has shown any ability to lyse Gram-positive bacteria is the combination of biological enzymatic lysis with chemical alkaline lysis, as disclosed in Liao et al., “Development of an Advanced Electrochemical DNA Biosensor for Bacterial Pathogen Detection”, J. Molec. Diag. 2007; 9(2):158-168 which has been incorporated herein by reference in its entirety. There are major drawbacks to enzymatic lysing methods however, including the time involved and lack of specificity of the enzymes.

Notwithstanding the above advances in the art, there is still room for improvement.

Accordingly, it would be desirable to have a means for lysing Gram-positive organisms sufficiently for electrochemical detection of target molecules. It would also be desirable if such lysing methods were less time intensive and more cost effective than previously utilized enzymatic lysis methods.

### SUMMARY

It is an object of the present invention to obviate or mitigate at least one of the above-mentioned disadvantages of the prior art.

It is another object of the present invention to provide a novel method for the universal lysis of a biological sample containing a variety of cell types, such that the cells are sufficiently lysed for detection (e.g., electrochemical detection) of target molecules within the cells, regardless of the cell type.

Accordingly, in one of its aspects, the present invention provides a method for extracting a target chemical compound from a cellular material in a sample, the method comprising the steps of:
(a) subjecting the sample to mechanical lysis to cause disruption of a cellular membrane in the cellular material;
(b) contacting the sample with an alkaline material to produce a lysate composition comprising the target chemical compound; and
(c) recovering the lysate composition from the sample.

In another of its aspects, the present invention provides a method for producing a lysate composition comprising RNA from a sample of mammalian origin comprising a cellular material, the method comprising the steps of:
(a) rotating a microfluidic centrifugal disk comprising a lysis chamber containing the sample;
(b) subjecting the sample to mechanical lysis to cause disruption of a cellular membrane in the cellular material; and
(c) contacting the sample in the lysis chamber with an alkaline solution to produce the lysate composition.

In yet another of its aspects, the present invention provides a method for extracting a nucleic acid from a cellular material in a sample comprising a bodily fluid or an inoculant derived therefrom, the method comprising the steps of:
(a) subjecting the sample to a first lysing process comprising mechanical lysis to cause disruption of a cellular membrane in the cellular material;
(b) subjecting the sample to a second lysing process comprising at least one of physical lysis, chemical lysis, biological lysis and any combination of two or more of these to produce a lysate composition comprising the nucleic acid; and
(c) recovering the lysate composition from the sample.

Accordingly, as described herein below, the present inventors have developed a method of lysis that is capable of extracting a target chemical compound from a cellular material (e.g., a nucleic acid from a biological sample containing Gram-negative, Gram-positive cells and other eukaryotic cells such as fungi), such that the target chemical compound may be detected using a hybridization detection assay (e.g., electrochemical detection). The present method involves a combination of mechanical lysis and non-mechanical lysis, where the non-mechanical lysis is preferably chemical alkaline lysis. While not wishing to be bound by any particular theory or mode of action, it is believed that the shearing forces from mechanical lysis make it possible to disrupt the thicker cell walls of the cellular material (e.g., Gram-positive cells, fungi and the like) and to facilitate extraction of the target chemical compound (e.g., a nucleic acid such as RNA), ideally without disrupting the target chemical compound (e.g., the signature sequence of the target nucleic acid) in the cellular material. The use of mechanical lysis alone is insufficient to allow for extraction of the target chemical compound from the cellular material, particularly when the method is applied to broad-based assay where it may not be known in advance whether the particular cellular material is actually present in the sample. For example, it may not be known in advance whether the sample contains the target chemical compound in the cellular material (e.g. it may not be known if the sample contains one or more of Gram-negative bacteria, Gram-positive bacteria or eukaryotic cells such as fungi). One of the advantages of the present method is that it has broad-based applicability for use with a sample containing one or both of Gram-negative and Gram-positive bacteria (the latter are particularly difficult to lyse using only chemical lysis techniques). When the target chemical compound is a nucleic acid such as RNA (e.g., ribosomal RNA or rRNA), chemical alkaline lysis will serve to denature the ribosomal complex—revealing the ribosomal RNA—and prepare the rRNA for detection in a hybridization detection assay (e.g., electrochemical detection).

As illustrated through experimental data hereinbelow, the present inventors have shown that combining mechanical lysis and chemical alkaline lysis is an effective method for extracting and preparing a target chemical compound (e.g., RNA such as rRNA) from a cellular material such as Gram-negative cells, Gram-positive cells and fungi cells sufficiently for assay detection (e.g. electrochemical detection) of the target chemical compound. The present method may be regarded as a general lysis method that has the potential to be used in a number of clinical applications, including species-specific detection of uropathogens in clinical urine specimens.

### BRIEF DESCRIPTION OF THE DRAWINGS

Embodiments of the present invention will be described with reference to the accompanying drawings, wherein like reference numerals denote like parts, and in which:

FIG. 1 is a preferred embodiment of an apparatus for use in carrying out mechanical lysis comprising a spin platform (left) and centrifugal disk (right);

FIG. 2 illustrates improved cell lysis using a combination of mechanical lysis and non-mechanical lysis;

FIG. 3 illustrates improved cell lysis using a combination of mechanical lysis and non-mechanical lysis for a broad variety of Gram-positive bacteria;

FIG. 4 illustrates optimal signal with a combination of mechanical lysis (OmniLyse®) plus NaOH for Gram-positive bacteria;

FIG. 5 illustrates improved signal with a combination of mechanical lysis (OmniLyse®) plus NaOH for a broad variety of Gram-positive bacteria;

FIG. 6 illustrates rRNA detection for various NaOH concentrations and mechanical lysis durations;

FIG. 7 illustrates Luminex signal after NaOH treatment from 0 to 5 minutes following a 1-minute mechanical lysis (OmniLyse®).

FIG. 8 illustrates a comparison of different enzyme concentrations when used in biological lysis of Gram-positive cells.

FIG. 9A illustrates a comparison of differing lengths of time of mechanical lysis (OmniLyse®) in combination with alkaline lysis.

FIG. 9B illustrates a comparison of different concentrations of NaOH in combination with mechanical lysis (OmniLyse®).

FIG. 10 illustrates the Luminex signal after lysing certain types of cells, including Gram-negative cells, Gram-positive cells, and yeast cells.

FIG. 11 illustrates the effect of different buffers used to neutralize a cell lysate.

### DETAILED DESCRIPTION

The present invention relates to a method for extracting a target chemical compound from a cellular material in a sample, the method comprising the steps of (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample. The method may further comprise neutralizing the sample by contacting the sample with a buffer solution. The method may further comprise contacting the sample with a nuclease inhibitor. The method may further comprise detecting at least one nucleotide sequence in the cell lysate.

Preferred embodiments of this method may include any one or a combination of any two or more of any of the following features:
the target chemical compound is a nucleic acid;
the target chemical compound is deoxyribonucleic acid (DNA);
the target chemical compound is ribonucleic acid (RNA);
the target chemical compound is ribosomal RNA (rRNA);
the rRNA is selected from the group consisting of 16S rRNA, 23S rRNA and any mixture thereof;
the rRNA is pre-ribosomal RNA;
the rRNA is mature rRNA.
the alkaline material is an alkaline solution;
the alkaline solution is sodium hydroxide;
the alkaline solution has a concentration of 10M or less, or 1M to 5M, or 1.5M to 3M, or 2M, or 3M;
the cellular material is an unknown cellular material;
the cellular material comprises a microorganism;
the cellular material comprises bacteria;
the cellular material comprises prokaryotic cells;
the cellular material comprises Gram-negative bacteria;
the cellular material comprises Gram-positive bacteria;
the cellular material comprises virally infected cells;
the cellular material comprises fungus cells;
the cellular material comprises yeast cells;
the sample comprises mammalian cellular material;
the sample comprises human cellular material;
the sample comprises a bodily fluid or an inoculant derived therefrom;
the bodily fluid is selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these;
after disruption of the cellular membrane in the cellular material, the sample is subjected to biological lysis;
after disruption of the cellular membrane in the cellular material, the sample is contacted with an enzyme;
the enzyme is selected from the group consisting of lysozyme, lysostaphin and any mixture thereof;
after disruption of the cellular membrane in the cellular material, the sample is subjected to physical lysis;
the physical lysis is selected from the group consisting of lysis is selected from the group of heating, osmotic shock, cavitation or any combination of two or more of these;
step (a) is conducted for a period of 10 minutes or less, or from 30 seconds to 10 minutes, or from 1 minute to 8 minutes, or for a period of time from 2 minutes±30 seconds, or 3 minutes±30 seconds, or 4 minutes±30 seconds, or 5 minutes±30 seconds, or 6 minutes±30 seconds, or 7 minutes±30 seconds;
the mechanical lysis is selected from the group consisting of French press, shaking, grinding, bead beating, centrifugation and any combination of two or more of these;
bead beating comprises beating with ceramic beads, glass beads, zirconium beads, silica-zirconium beads, steel beads or any combination of two or more of these;
bead beating comprises the use of magnetic beads;
the mechanical lysis comprises using OmniLyse® or a functional equivalent thereof;
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
centrifugation is carried out on a centrifugal disk;
steps (a) and (b) are carried out concurrently;
steps (a) and (b) are carried out sequentially;
step (b) is carried out after commencement of disruption of the cellular membrane in step (a);
the buffer solution is a phosphate buffer solution;
the buffer solution has a pH of less than 7, or a pH in the range of 5 to 7.5, or a pH in the range of 6 to 7.
the sample is contacted with a nuclease inhibitor prior to step (a);
the nuclease inhibitor is an RNAse inhibitor;
at least one nucleotide sequence in the cell lysate may be detected using a sandwich assay;
the sandwich assay may be conducted on an electrochemical sensor platform;
at least one nucleotide sequence in the cell lysate may be detected using an electrochemical sensor platform;
at least one nucleotide sequence in the cell lysate may be detected by contacting the cell lysate with a capture probe;
at least one nucleotide sequence in the cell lysate may be detected by contacting the cell lysate with a magnetic bead;
the magnetic bead comprises a capture probe;
the capture probe comprises one or more nucleic acids;
at least one nucleotide sequence in the cell lysate may be detected by contacting the cell lysate with a detector probe;
the detector probe comprises one or more nucleic acids;
the one or more nucleic acids of the capture probe or detector probe comprise one or more deoxyribonucleic acid (DNA);
the one or more nucleic acids of the capture probe or detector probe comprise one or more peptide nucleic acids (PNAs);
the one or more nucleic acids of the capture probe or detector probe comprise one or more locked nucleic acids (LNAs);
the detector probe comprises a detectable label.

In another of its aspects, the present invention relates to a method for producing a lysate composition comprising RNA from a sample of mammalian origin comprising a cellular material, the method comprising the steps of (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition.

Preferred embodiments of this method may include any one or a combination of any two or more of any of the following features:
the RNA is ribosomal RNA (rRNA);
the rRNA is selected from the group consisting of 16S rRNA, 23S rRNA and any mixture thereof;
the rRNA is pre-ribosomal RNA;
the rRNA is mature rRNA.
the alkaline solution is sodium hydroxide;
the alkaline solution has a concentration of 10M or less, or 1M to 5M, or 1.5M to 3M, or 2M, or 3M;
the sample comprises human cellular material;
the sample comprises a bodily fluid or an inoculant derived therefrom;
the bodily fluid is selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these;
steps (a) and (b) are conducted for a period of 10 minutes or less, or from 30 seconds to 10 minutes, or from 1 minute to 8 minutes, or for a period of time from 2 minutes±30 seconds, or 3 minutes±30 seconds, or 4 minutes±30 seconds, or 5 minutes±30 seconds, or 6 minutes±30 seconds, or 7 minutes±30 seconds
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
centrifugation is carried out on a centrifugal disk;
steps (a) and (b) are carried out concurrently;
steps (b) and (c) are carried out concurrently;
steps (b) and (c) are carried out sequentially; or
step (c) is carried out after commencement of disruption of the cellular membrane in step (b).

In another of its aspects, the present invention relates to a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample.

Preferred embodiments of this method may include any one or a combination of any two or more of any of the following features:
the nucleic acid comprises ribosomal RNA (rRNA);
the rRNA is pre-ribosomal RNA;
the rRNA is selected from the group consisting of 16S rRNA, 23S rRNA and any mixture thereof;
the rRNA is mature rRNA;
the chemical lysis comprises contacting the bodily fluid with an alkaline solution;
the alkaline solution comprises a sodium hydroxide solution;
the alkaline solution has a concentration of 10M or less, or 1M to 5M, or 1.5M to 3M, or 2M, or 3M;
the sample comprises human cellular material;
the human cellular material is a bodily fluid or an inoculant derived therefrom;
the bodily fluid is selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these;
step (a) is conducted for a period of 10 minutes or less, or from 30 seconds to 10 minutes, or from 1 minute to 8 minutes, or for a period of time from 2 minutes±30 seconds, or 3 minutes±30 seconds, or 4 minutes±30 seconds, or 5 minutes±30 seconds, or 6 minutes±30 seconds, or 7 minutes±30 seconds;
the mechanical lysis comprises a combination of centrifugation and puck lysing;
the mechanical lysis comprises a combination of centrifugation and magnetic puck lysing;
the combination of centrifugation and puck lysing is carried out in a common lysis chamber;
steps (a) and (b) are carried out concurrently;
steps (a) and (b) are carried out sequentially; or
step (b) is carried out after commencement of disruption of the cellular membrane in step (a).

As used herein, certain terms may have the following defined meanings.

As used in the specification and claims, the singular form “a,” “an” and “the” include singular and plural references unless the context clearly dictates otherwise. For example, the term “a cell” includes a single cell as well as a plurality of cells, including mixtures thereof.

In one of its aspects, the present invention provides methods for extracting a target chemical compound from a cellular material in a sample. The methods may comprise: subjecting the sample to mechanical lysis to cause disruption of a cellular membrane in the cellular material; contacting the sample with an alkaline material to produce a lysate composition comprising the target chemical compound; and recovering the lysate composition from the sample.

Provided in one embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the target chemical sample may be a nucleic acid. In some embodiments, the nucleic acid may be deoxyribonucleic acid (DNA). Examples of RNA involved in protein synthesis include, but are not limited to, messenger RNA (mRNA), transfer RNA (tRNA), transfer-messenger RNA (tmRNA), single recognition particle RNA (SRP RNA), and ribosomal RNA (rRNA). In some embodiments, the nucleic acid may be ribonucleic acid (RNA). In certain preferred embodiments, the nucleic acid may be ribosomal RNA (rRNA), or more preferably may pre-ribosomal rRNA, mature rRNA, or may be selected from the group consisting of 16S rRNA, 23S rRNA or any mixture thereof.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein step (b) may comprise contacting the cellular material in the sample with an alkaline solution. In some embodiments, the alkaline solution may be a sodium hydroxide solution. In certain preferred embodiments, the alkaline solution may have a concentration of about 10M or less, preferably of about 1M to 5M, and more preferably of about 1.5M to 3M. In certain preferred embodiments, the alkaline solution may have a concentration of about 2M. In other preferred embodiments, the alkaline solution may have a concentration of about 3M.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be an unknown cellular material.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be either a microorganism, prokaryotic cells, virally infected cells, fungus cells, or yeast cells. Examples of yeast cells may include but are not limited to Candida cells. Methods for detecting the presence of a fungal organisms within a biological sample, such as yeast have been disclosed in International Patent Publication No. WO 2013166460 and WO 2015013324, both of which are incorporated herein by reference herein in their entirety.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the cellular material may be bacteria. In certain preferred embodiments, the bacteria may be Gram-negative bacteria, Gram-positive bacteria, or a mixture thereof. Examples of Gram-negative bacteria may include, but are not limited to Escherichia coli, Salmonella, Shigella, Enterobaceriaceae, Pseudomonas, Moraxella, Helicobacter, Strenotrophomonas, Bdellovibrio, and Legionella. Examples of Gram-positive bacteria may include, but are not limited to Enterococcus, Staphylococcus, Streptococcus, Actinomyces, Bacillus, Clostridium, Corynebacterium, Listeria, and Lactobacillus.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the sample may comprise mammalian cellular material, preferably human cellular material, and more preferably a bodily fluid or an inoculant derived therefrom. In certain preferred embodiments, the bodily fluid may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these. Other examples of mammalian cellular material include but are not limited to samples from monkeys, cats, dogs, sheep, goats, cows, pigs, horses, or rabbits.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein after disruption of the cellular membrane in the cellular material, the sample may be subjected to biological lysis. In some embodiments, the biological lysis may include contacting the sample with an enzyme. In certain preferred embodiments, the enzyme may be selected from the group consisting of lysozyme, lysostaphin and any mixture thereof.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein after disruption of the cellular membrane in the cellular material, the sample may be subjected to physical lysis. In some embodiments, the physical lysis may be selected from the group consisting of heating, osmotic shock, cavitation or any combination of two or more of these. Physical lysis methods such as those mentioned above are common in the art. For example, lysis by heating may comprise placing the sample in a water bath, heat block, or temperature controlled container, where the temperature of the water bath, heat block, or temperature controlled container may be less than or equal to about 100o C, preferably between about 40° C. and about 100° C., or more preferably the sample may be heated at 45° C., 50° C., 55° C., 60° C., 65° C., 70° C., 75° C., 80° C., 85° C., 90° C., or 95° C. Cavitation may comprise nitrogen cavitation which may be performed by (a) placing cells from a sample in a pressure vessel; (b) dissolving oxygen-free nitrogen in the cells under high pressure; and (c) releasing the pressure in the vessel. Osmotic shock may be performed by changing the concentration of a salt, substrate or solute around cells from a sample, such that the cells rupture and/or release intracellular materials, such as nucleic acid molecules and proteins.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein step (a) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may be selected from the group consisting of French press, shaking, grinding, bead beating, centrifugation and any combination of two or more of these. For example, lysis by French press may performed by passing a sample through a narrow valve under high pressure. Lysis by grinding may be performed by placing a sample in a grinder. Examples of grinders may include, but are not limited to, a ball mill, coffee grinder, Geno/Grinder, and Retsch Mixer Mill. A ball mill for instance, may comprise a hollow cylindrical shell and one or more balls, where the balls may be made of chrome steel, stainless steel, ceramic, or rubber. Lysis by grinding may comprise, for example, the use of a mortar and pestle. Lysis by shaking may comprise, for example, mixing the sample with some sort of bead or matrix, and placing the sample on a violent high-speed shaker.

In some embodiments, where the mechanical lysis is performed by bead beating, said bead beating my comprise beating the sample with ceramic beads, glass beads, zirconium beads, silica-zirconium beads, steel beads or any combination of two or more of these. In certain preferred embodiments, bead beating may comprise the use of magnetic beads. By way of non-limiting example, silica-zirconium beads may be preferable for use in the disclose inventions as they are chemically inert and have been shown not to interfere with the assay techniques.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may comprise using OmniLyse® or a functional equivalent thereof. Mechanic lysis with OmniLyse® or a functional equivalent thereof, for instance, may comprise the use of a small chamber containing, for example, zirconium beads, where the chamber is then connected to a syringe and a motor. By way of non-limiting example, OmniLyse® lysis may comprise drawing a solution into the chamber with the syringe and turning on the motor to move the beads around at around 30,000 rpm with a small propeller, then ejecting the solution back into a tube using the syringe.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the mechanical lysis may comprise a combination of centrifugation and puck lysing. In some embodiments, the puck lysing may be magnetic puck lysing. In certain preferred embodiments, the combination of centrifugation and disk lysing may be carried out in a common lysis chamber, where preferably centrifugation and puck lysing may be carried out on a centrifugal disk (CD). By way of non-limiting example, the centrifugal disk may comprise one or more microfluidic lysis chambers connected to one another by one or more microfluidic channels, where at least one of the microfluidic lysis chambers has an inlet port which may be configured to receive a fluid sample. Each lysis chamber of the CD may contain one or more magnetic lysis pucks and a series of beads, wherein the lysis pucks and beads are small enough to be able to move within the lysis chamber, but not small enough to exit the lysis chamber through any of the microfluidic channels. The CD may be configured to fit on a rotating platform connected to a motor, such that when the CD is placed on the platform and the motor is turned on, the CD will rotate. The platform my further comprise a series of stationary magnets which may be configured such that when the CD is rotating, the interaction between the stationary magnets and the magnetic lysis pucks causes the lysis pucks to move back and forth within each of the one or more lysis chambers. Lysis methods such as this are known in the art, including those disclosed in U.S. Pat. No. 8,303,911 which is incorporated by reference herein in its entirety.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein steps (a) and (b) may be carried out concurrently.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein steps (a) and (b) may be carried out sequentially. In certain preferred embodiments, step (b) may be carried out after commencement of disruption of the cellular membrane in step (a). This sequential method may be preferred because alkaline lysing alone will not be able to disrupt the cellular membrane of Gram-positive cells and/or fungal cells. Thus, in order to get access to the target compound within a Gram-positive and/or fungal cell, the cellular membrane must first be disrupted by the shear forces of mechanical lysing.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises neutralizing the sample by contacting the sample with a buffer solution. When a sample is contacted with an alkaline solution, high concentrations of hydroxide ions break apart the protein components of a cell ribosome, unwind the secondary structure of rRNA, and break it into pieces. If this process is left unchecked, it will eventually break down the entire rRNA into single bases. In order to arrest this process, a concentrated buffer solution may be added to neutralize the pH of the lysate. In some embodiments, the buffer solution may be a phosphate buffer solution. In certain preferred embodiments the buffer solution may have a pH of less than 7, preferably in the range of about 5 to 7.5, and more preferably in the range of 6 to 7.

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises contacting the sample with a nuclease inhibitor. In some embodiments, the sample may be contacted with a nuclease inhibitor prior to step (a). In certain preferred embodiment, the nuclease inhibitor may be an RNAse inhibitor. For example, the RNAse inhibitor may be selected from but is not limited to 2′-cytidine monophosphate free acid (2′-CMP), aluminon, adenosine 5′-pyrophosphate, 5′-diphosphoadenosine 3′-phosphate (ppA-3′-p), 5′-diphosphoadenosine 2′-phosphate (ppA-2′-p), Leucine, poly-L-aspartic acid, tyrosine-glutamic acid polymer, oligovinysulfonic acid, 5′-phospho-2′-deoxyuridine 3′-pyrophosphate P′→5′-ester with adenosine 3′-phosphate (pdUppAp).

Provided in another embodiment is a method for extracting a target chemical compound from a cellular material in a sample, the method comprising (a) subjecting the sample to mechanical lysis to cause disruption of a cellular membrane in the cellular material; (b) contacting the sample with an alkaline material to produce a lysate composition comprising the target chemical compound; and (c) recovering the lysate composition from the sample, wherein the method further comprises detecting at least one nucleotide sequence in the cell lysate. In some embodiments, one or more nucleotide sequence may be detected using a sandwich assay, preferably where the sandwich assay is conducted on an electrochemical sensor platform. In certain preferred embodiments, one or more nucleotide sequences may be detected by contacting the cell lysate with a capture probe. In other preferred embodiments, one or more nucleotide sequences may be detected by contacting the cell lysate with a magnetic bead, preferably where the magnetic bead comprises a capture probe or a detector probe. In certain preferred embodiments, the capture probe or detector probe may comprise one or more nucleic acids, examples of which may include but are not limited to DNA, peptide nucleic acids (PNAs), locked nucleic acids (LNAs) or any combination thereof. By way of non-limiting example, the capture probes and detector probes may each comprise 1, 2, 3, 4, 5, 6, 7, 8, 9, 10, 11, 12, 13, 14, 15, 16, 17, 18, 19, 20 or more nucleic acids. In further preferred embodiments, the detector probe may comprise a detectable label. By way of non-limiting example, the detectable label may be selected from a radionuclide, an enzymatic label, a chemiluminescent label, a hapten, and a fluorescent label. A fluorescent label for example, may be a fluorescent molecule selected from a fluorophore, a cyanine dye, and a near infrared (NIR) dye, or more preferably the fluorescent molecule may be fluorescein or fluorescein isothiocyanate (FITC). A hapten label may for example be selected from DCC, biotin, nitropyrazole, thiazolesulfonamide, benzofurazan, and 2-hydroxyquinoxaline.

In another of its aspects, the present invention provides a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition.

Provided in one embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the RNA may pre-ribosomal RNA, mature RNA, or may be selected from the group consisting of 16S rRNA, 23S rRNA or any mixture thereof.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the alkaline solution may comprise a sodium hydroxide solution. In certain preferred embodiments, the alkaline solution may have a concentration of about 10M or less, preferably of about 1M to 5M, and more preferably of about 1.5M to 3M. In certain preferred embodiments, the alkaline solution may have a concentration of about 2M. In other preferred embodiments, the alkaline solution may have a concentration of about 3M.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the sample may comprise human cellular material, preferably a bodily fluid or an inoculant derived therefrom. In certain preferred embodiments, the bodily fluid may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (a) and (b) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (a) and (b) may be carried out concurrently.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (b) and (c) may be carried out concurrently.

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein steps (b) and (c) may be carried out sequentially. In certain preferred embodiments, step (c) may be carried out after commencement of disruption of the cellular membrane in step (b).

Provided in another embodiment is a method for producing a lysate composition comprising RNA from a sample of mammalian origin comprising a cellular material, the method comprising the steps of: (a) rotating a microfluidic centrifugal disk comprising a lysis chamber containing the sample; (b) subjecting the sample to mechanical lysis to cause disruption of a cellular membrane in the cellular material; and (c) contacting the sample in the lysis chamber with an alkaline solution to produce the lysate composition, wherein the mechanical lysis may comprise a combination of centrifugation and puck lysing. In some embodiments, the puck lysing may be magnetic puck lysing. In certain preferred embodiments, the combination of centrifugation and puck lysing may be carried out in a common lysis chamber, preferably centrifugation and puck lysing may be carried out on a centrifugal disk.

In yet another of its aspects, the present invention provides a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample.

Provided in one embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the nucleic acid may be deoxyribonucleic acid (DNA) or ribonucleic acid (RNA). In certain preferred embodiments, the nucleic acid may be ribosomal RNA, or more preferably may pre-ribosomal RNA, mature RNA, or may be selected from the group consisting of 16S rRNA, 23S rRNA or any mixture thereof.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the chemical lysis may comprise contacting the sample with an alkaline solution. In some embodiments, the alkaline solution may comprise a sodium hydroxide solution. In certain preferred embodiments, the alkaline solution may have a concentration of about 10M or less, preferably of about 1M to 5M, and more preferably of about 1.5M to 3M. In certain preferred embodiments, the alkaline solution may have a concentration of about 2M. In other preferred embodiments, the alkaline solution may have a concentration of about 3M.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the bodily fluid may comprise human cellular material, and more preferably may be selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein step (a) may be conducted for a period of about 10 minutes or less, preferably from about 30 seconds to about 10 minutes, more preferably from about 1 minute to 8 minutes, and most preferably for a period of about 2 minutes±30 seconds, about 3 minutes±30 seconds, about 4 minutes±30 seconds, about 5 minutes±30 seconds, about 6 minutes±30 seconds, or about 7 minutes±30 seconds.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein the mechanical lysis may comprise a combination of centrifugation and puck lysing. In some embodiments, the puck lysing may be magnetic puck lysing. In certain preferred embodiments, the combination of centrifugation and puck lysing may be carried out in a common lysis chamber, preferably centrifugation and puck lysing may be carried out on a centrifugal disk.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein steps (a) and (b) may be carried out concurrently.

Provided in another embodiment is a method for extracting a nucleic acid from a cellular material in a sample comprising a bodily fluid or an inoculant derived therefrom, the method comprising the steps of (a) subjecting the sample to a first lysing process comprising mechanical lysis to cause disruption of a cellular membrane in the cellular material; (b) subjecting the sample to a second lysing process comprising at least one of physical lysis, chemical lysis, biological lysis and any combination of two or more of these to produce a lysate composition comprising the nucleic acid; and (c) recovering the lysate composition from the sample, wherein steps (a) and (b) may be carried out sequentially. In certain preferred embodiments, step (b) may be carried out after commencement of disruption of the cellular membrane in step (a).

The methods disclose herein may comprise performing one or more mechanical lyses and one or more non-mechanical lyses.

### EXPERIMENTAL EXAMPLES

Embodiments of the present invention will now be illustrated with reference to the following examples which should not be used to construe or limit the scope of the present invention.

### Example 1. Cell Lysis Using Mechanical and Non-Mechanical Lysis

In this Example, the materials and methods for lysing bacteria (e.g., Staphylococcus aureus) using mechanical lysis (OmniLyse® or centrifugal disk) and non-mechanical lysis (NaOH) are provided.

### Materials

The following materials were used:
1. OmniLyse® Lysis Kit. Available from ClaremontBio.com: http://www.claremontbio.com/OmniLyse_Cell_Lysis_Kits_s/56.htm;
2. 1.7 ml microcentrifuge tubes;
3. mixture of identification (ID) detector probes (100 nM) in 1 M phosphate buffer pH 6.4;
4. 96-well plate containing Luminex MTAG beads functionalized with capture probes;
5. 1× Tm HB=0.1 M Tris pH 8.0, 0.2 M NaCl, 0.08% Triton X-100;
6. 1 M NaOH; and
7. Streptavidin-phycoerythrin conjugate.

### Equipment

The following equipment was used:
1. Shaker Incubator;
2. Biotek 405TS Plate Washer; and
3. Luminex MagPix Assay System.

### Method 1: OmniLyse® and NaOH

The following methodology were used:
1. The OmniLyse® cartridges were pre-wetted by filling the cartridge with filter-sterilized superwater, and emptying with the syringe plunger. This step was repeated one additional time. One OmniLyse® cartridge was needed for each specimen and control.
2. 40 μl of 1 M NaOH was added to 1.7 ml microcentrifuge tubes. 2 extra tubes were included for negative and positive controls.
3. 80 μl of specimen was added to a microcentrifuge tube that contained 40 μml M NaOH and mixed by pipetting.
4. The syringe plunger was used to draw 120 μl of specimen+NaOH from the sample tube into the OmniLyse® cartridge. The OmniLyse® cartridge was turned on for 1 minute.
5. After OmniLyse® treatment, the plunger was used to dispense up to 120 μl of lysate into a tube and incubated at room temperature to complete the 5 minutes of exposure to NaOH.
6. The lysates were neutralized by adding 100 μl of ID detector probe mixture to each tube and mixed by pipetting.
7. 190 μl of neutralized lysate was added to wells in the 96-well ID plate. Negative and positive control lysates were also added.
8. The plate was shaken (without magnet) for 15 minutes on the variable setting with the Biotek plate washer.
9. The beads were washed in the Biotek plate washer using the Biotek Bead Washing Protocol below.
10. While the plate was washing, 2 μl of 1 mg/ml Streptavidin-PE stock was added to 1000 1× Tm HB to yield 2 μg/ml.
11. After the plate was finished washing, 75 μl of 2 μg/ml Streptavidin-PE was added to the appropriate wells.
12. The plate was shaken on variable speed with the Biotek plate washer for 1 minute.
13. The beads were washed with the Biotek plate washer following the protocol listed below.

The beads were then measured in the Luminex MagPix instrument.

Method 2: Centrifugal disk and NaOH

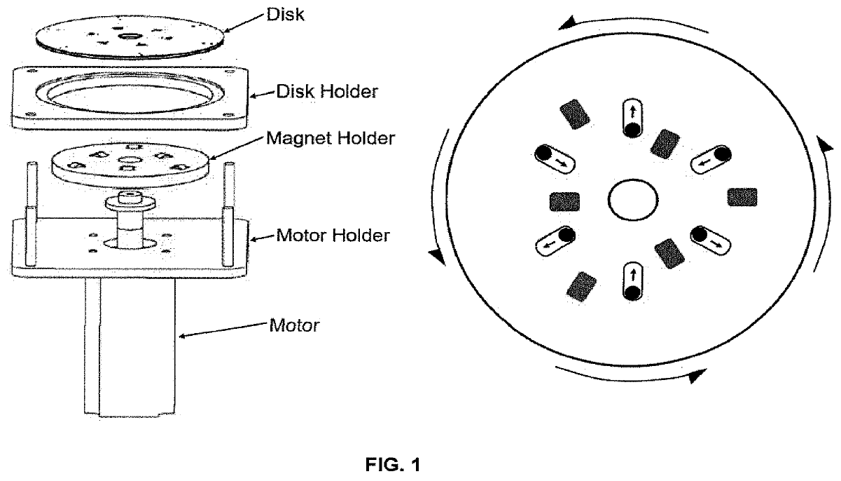

The method for performing mechanical lysis using a centrifugal disk is similar to Method 1 described above, except that the OmniLyse in step 4 of Method 1 was replaced by a centrifugal disk containing a lysis chamber containing zirconium beads and a stainless-steel lysing puck (see FIG. 1). 120 μl of specimen and NaOH from step 3 of Method 1 was placed in the CD lysis chamber and the centrifugal disc was rotated at 100 rpm for 5 minutes. As the centrifugal disc rotated on the spin platform, magnets below the disc caused the stainless-steel lysing pucks to move back and forth in the lysis chamber, which when combined with zirconium beads provided grinding action.

Biotek Bead Washing Protocol (using 96-well plate magnet):
1. Shake on medium for 30 seconds
2. Soak for 30 seconds
3. Aspirate
4. Dispense 200 μl of 1× Tm HB per well
5. Shake on medium for 30 seconds
6. Soak for 30 seconds
7. Aspirate
8. Dispense 200 μl of 1× Tm HB per well
9. Shake on medium for 30 seconds
10. Soak for 30 seconds
11. Aspirate
12. FINAL WASH ONLY: Dispense 50 μl

Biotek 97 well plate washer settings:
1. Aspirate options—Z=43 (5.46 mm above carrier), X=30 (1.37 mm right of center)
2. Dispense options—Z=130 (16.52 mm above carrier), X=0
3. Slow mixing→7 Hz (420 rpm)
4. Medium mixing→13 Hz (780 rpm)
5. Fast mixing was performed at 19 Hz (1140 rpm).

Variable mixing comprised repeated cycles of slow, medium, and fast mixing at approximately 1.5 seconds each.

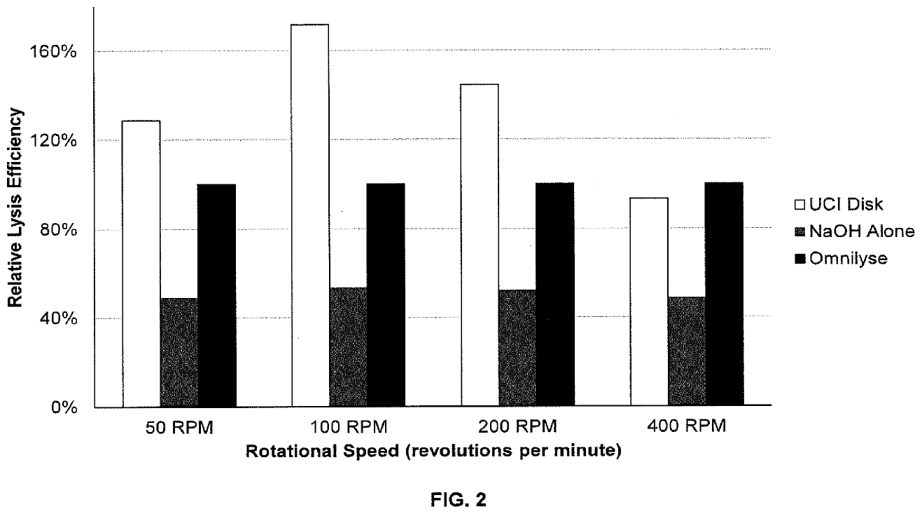

As shown in FIG. 2, the combination of mechanical lysis and non-mechanical lysis of Staphylococcus areus resulted in more efficient lysis than non-mechanical lysis with NaOH alone. FIG. 2 shows that at 50, 100 and 200 revolutions per minute (RPM), mechanical lysis with a centrifugal disk in combination with non-mechanical lysis using NaOH (first column) and mechanical lysis with OmniLyse® in combination with non-mechanical lysis using NaOH (third column) resulted in more efficient lysis compared to chemical lysis using NaOH alone (second column). The efficacy of the cell lysis was measured by detecting the quantity of rRNA released from identical samples.

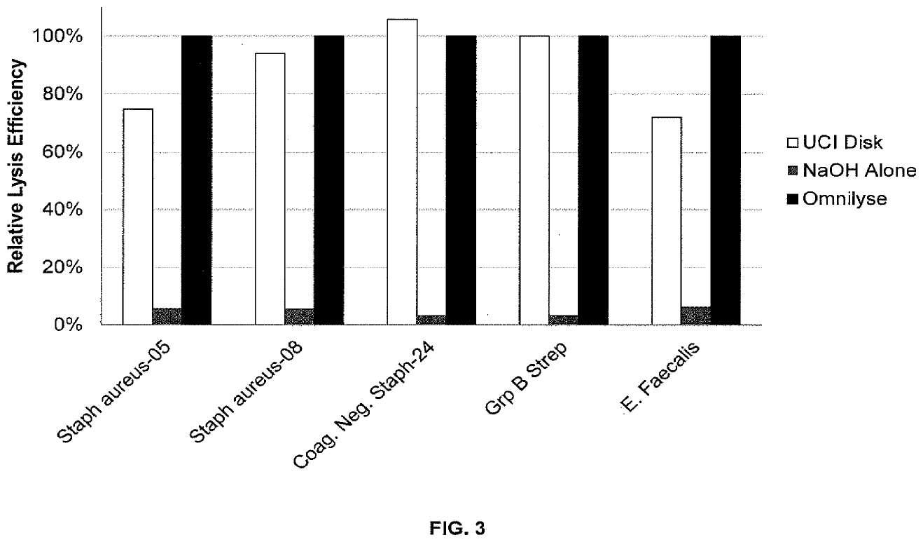

As shown in FIG. 3, mechanical lysis with a centrifugal disk in combination with non-mechanical lysis using NaOH (first column) and mechanical lysis with OmniLyse® in combination with non-mechanical lysis using NaOH (third column) resulted in more efficient lysis for a broad variety of Gram-positive bacteria compared to chemical lysis using NaOH alone (second column). The efficacy of the cell lysis was measured by detecting the quantity of rRNA released from identical samples.

### Example 2. Mechanical Lysis and Non-Mechanical Lysis of Gram-Positive Bacteria Results in More Efficient Detection of rRNA as Compared to a Combination of Enzymatic Lysis, Detergent Lysis and Chemical Lysis

In this Example, using the relevant materials and methodology described in Example 1, Gram-positive bacteria were lysed using a two-step lysis using either (a) Step 1: enzymatic lysis and detergent lysis, and Step 2: chemical lysis (e.g., Step 1: Triton X-100 and lysozyme, and Step 2: NaOH); or (b) Step 1: mechanical lysis and Step 2: chemical lysis (e.g., Step 1: OmniLyse® and Step 2: NaOH), followed by detection of rRNA using a Luminex® instrument.

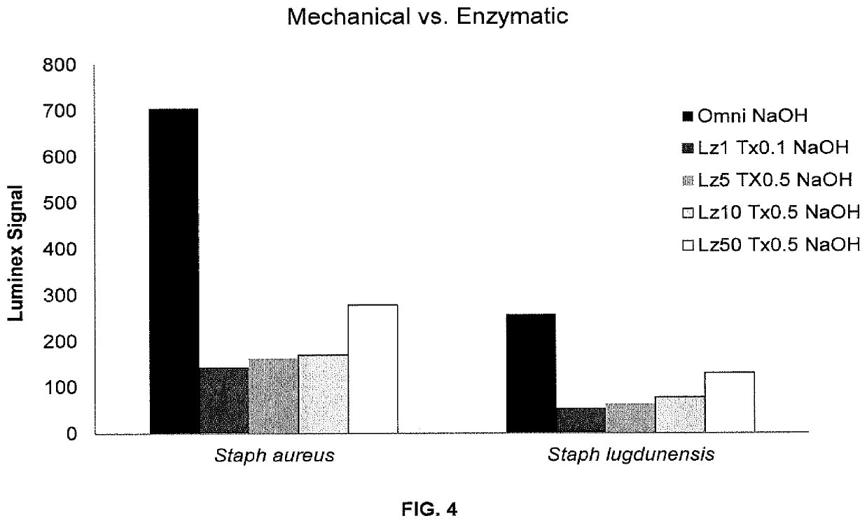

As shown in FIG. 4, the detection of rRNA was greatly increased following mechanical lysis using OmniLyse® in combination with chemical lysis using NaOH (first column) as compared to the detection of rRNA following enzymatic lysis using lysozyme and detergent lysis using Triton X-100 in combination with chemical lysis using NaOH.

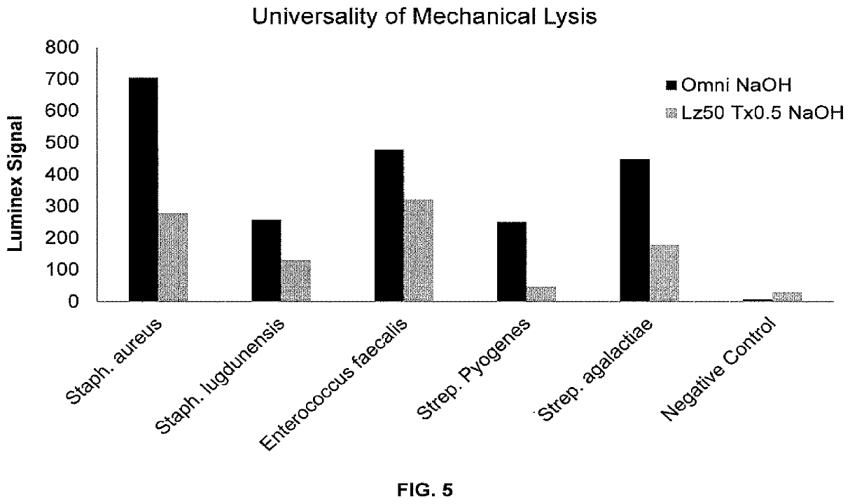

As shown in FIG. 5, mechanical lysis using OmniLyse® in combination with chemical lysis using NaOH (first column) resulted in improved detection of rRNA from a broad variety of Gram-positive bacteria (e.g., Staphylococcus aureus, Staphylococcus lugdunensis, Enterococcus faecalis, Streptococcus pyogenes, and Streptococcus Agalactiae) compared to enzymatic lysis using lysozyme and detergent lysis using Triton X-100 in combination with chemical lysis using NaOH.

These results demonstrate that the first step of enzyme plus detergent followed by NaOH treatment results in less efficient detection of rRNA from Gram-positive cells than the combination of mechanical lysis plus NaOH.

### Example 3. Impact of the Duration of Mechanical Lysis and Concentration of NaOH on rRNA Detection

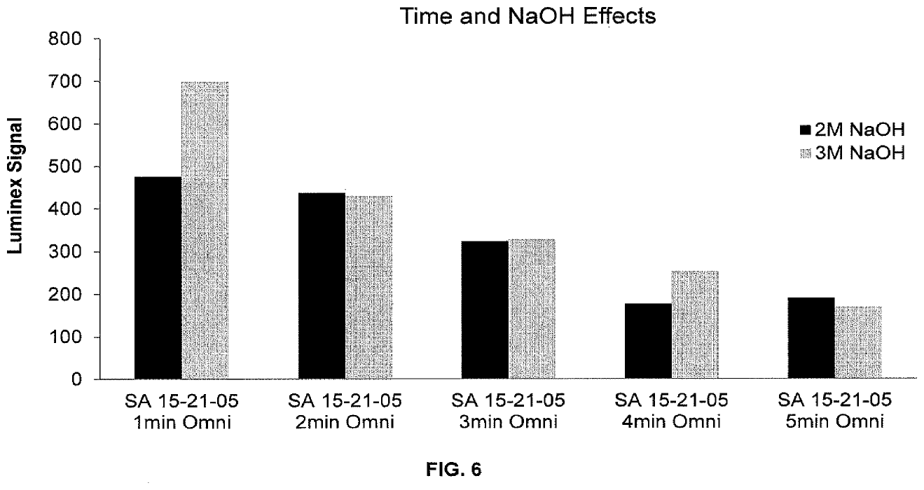

In this Example, using the relevant materials and methodology described in Example 1, the impact of the duration of mechanical lysis and concentration of NaOH on rRNA detection from Staphylococcus aureus was investigated. In the first step, bacteria were lysed for 1, 2, 3, 4, or 5 minutes using OmniLyse® and then chemically lysed using 2M NaOH or 3M NaOH for a duration of 5 minutes. As shown in FIG. 6, an optimal signal was achieved with mechanical lysis for 1 minute followed by chemical lysis using 3M NaOH.

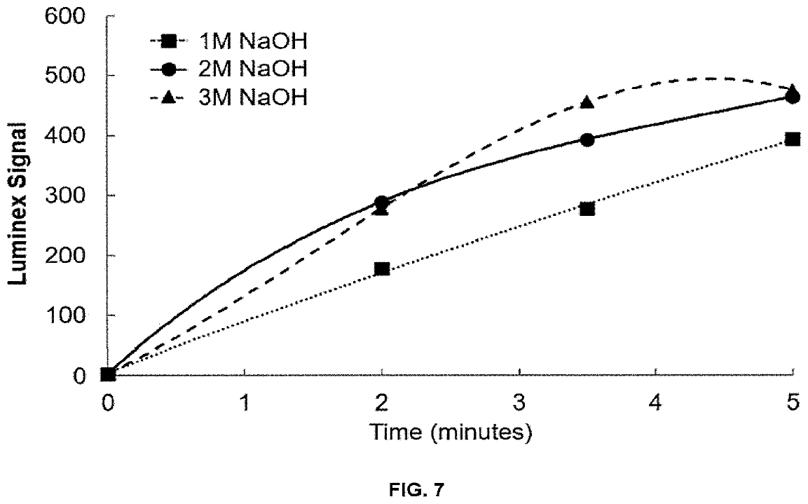

A separate experiment was performed to determine the optimal duration of NaOH treatment following a 1-minute mechanical lysis (OmniLyse®). For all NaOH concentrations, the optimal duration of NaOH treatment was found to be 5 minutes (FIG. 7).

### Example 4. Efficacy of Various Concentrations of Lysozyme Lysis Buffer on Gram-Positive Isolates

In step one of this example, the impact of biological (enzymatic in this case) lysis at different concentrations was investigated and compared to a combination of mechanical and alkaline lysis. During this experiment, a series of Gram-positive bacteria were lysed using different concentrations of lysozyme enzyme solution, either with or without the addition of 1-minute mechanical lysis (OmniLyse®). Following lysis, the cell lysate was contacted with specific capture probes and detector probes, using the relevant materials and methodology described in Example 1, to detect one or more nucleotide sequences in the cell lysate.

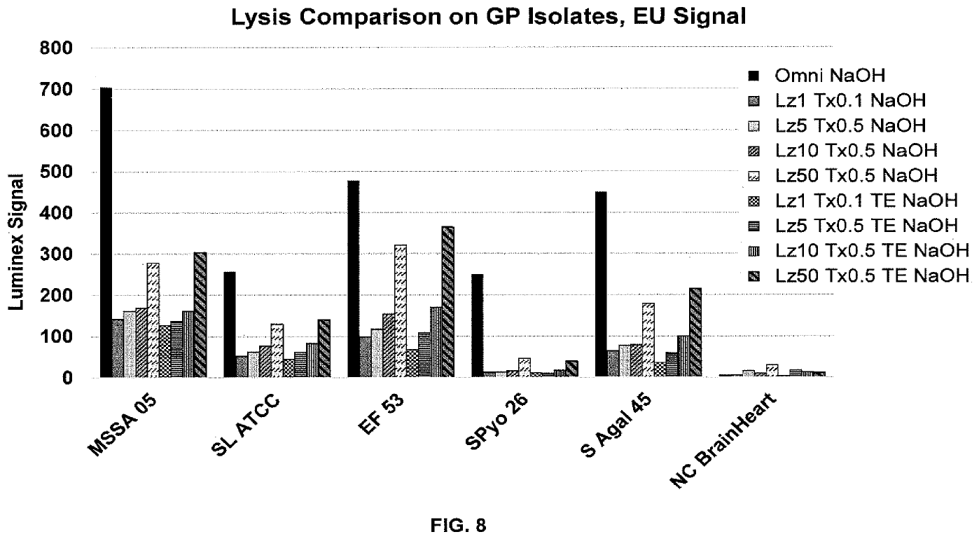

In step two, a separate experiment was performed, using the relevant materials and methodology described in Example 1, where Gram-positive bacteria were subjected to NaOH treatment following 1-minute mechanical lysis (OmniLyse®). The results for step one and step two were compared as shown in FIG. 8.

### Experimental Materials

The following materials were used:
1. OmniLyse® Lysis Kit. Available from ClaremontBio.com: http://www.claremontbio.com/OmniLyse_Cell_Lysis_Kits_s/56.htm;
2. Bacteria samples including: MSSA 15-21-05; Staph Lugdunensis ATCC; E. faecalis 07-09-53; Strep. pyogenes 15-21-26; and Strep. agalactiae 07-09-45
3. Lysis buffer including:
   (a) Lysozyme @ 1 mg/mL, Triton X-100 @ 0.1%, in H2O
   (b) Lysozyme @ 5 mg/mL, Triton X-100 @ 0.5%, in H2O
   (c) Lysozyme @ 10 mg/mL, Triton X-100 @ 0.5%, in H2O
   (d) Lysozyme @ 50 mg/mL, Triton X-100 @ 0.5%, in H2O
   (e) Lysozyme @ 1 mg/mL, Triton X-100 @ 0.1%, in 20 mM Tris-HCl 2 mM EDTA pH 8.0
   (f) Lysozyme @ 5 mg/mL, Triton X-100 @ 0.5%, in 20 mM Tris-HCl 2 mM EDTA pH 8.0
   (g) Lysozyme @ 10 mg/mL, Triton X-100 @ 0.5%, in 20 mM Tris-HCl 2 mM EDTA pH 8.0
   (h) Lysozyme @ 50 mg/mL, Triton X-100 @ 0.5%, in 20 mM Tris-HCl 2 mM EDTA pH 8.0
4. 96-well plate containing Luminex MTAG beads functionalized with capture probes; and
5. 1 M NaOH.

### Experimental Methods

The following experimental variables were used for the Lysozyme Buffer Set-Up:
1. The Lysozyme Buffers were made the same for every concentration, including:
   (a) 40 uL Bacteria+10 uL Enzymatic Lysis Buffer (5 min @ room temperature)
   (b) 25 uL 1M NaOH (5 min)
   (c) 75 uL 1M Phosphate Buffer

### Results

As shown in FIG. 8, the best enzymatic lysis condition used 50 mg/mL Lysozyme and 0.5% Triton X-100— i.e., 3(d) and 3(h) above.

### Example 5. Testing Relationship Between Strength of NaOH and Timing of OmniLyse®

### Experimental Methods

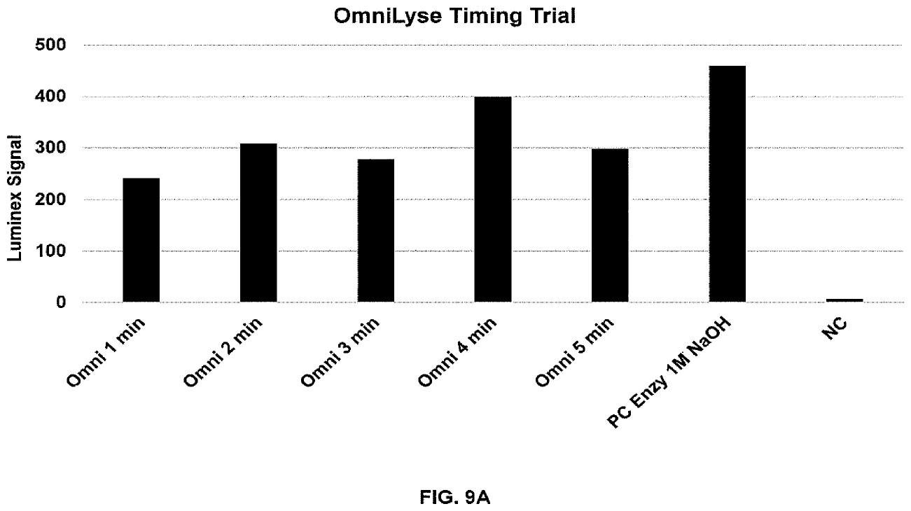

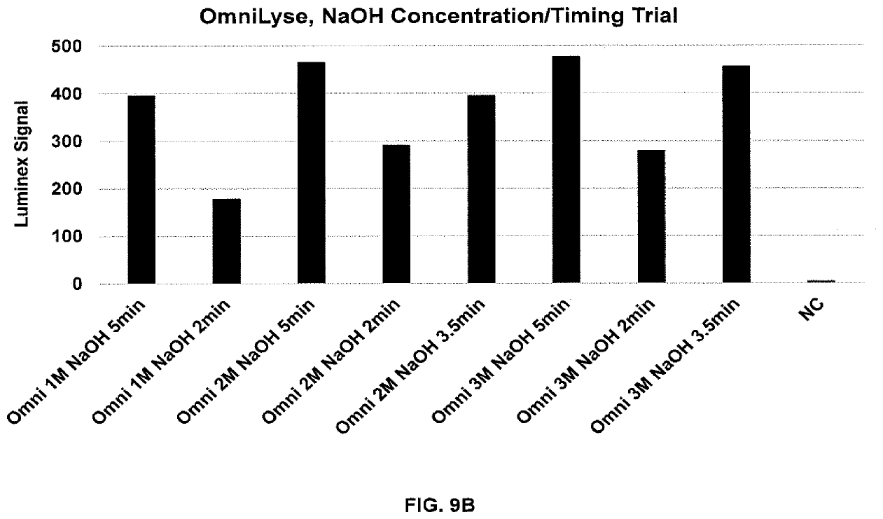

In this example, two experiments were performed. In the first experiment, using the relevant materials and methodology described in Example 1, the relationship between strength of NaOH and timing of Omnilyse® was investigated. In the first step, samples of Gram-positive bacteria (Staphylococcus aureus) were lysed for 1, 2, 3, 4, or 5 minutes using OmniLyse® and then chemically lysed using 1M NaOH for 5 minutes after OmniLyse® treatment. Results from this lysis were compared to enzymatic lysis as a control (See FIG. 9A)

In a second experiment, bacteria lysis of Gram-positive bacteria (Staphylococcus aureus) was performed with OmniLyse® for 2, 3.5 or 5 minutes with 1M, 2M or 3M NaOH (See FIG. 9B).

### Results

As shown in FIGS. 9A and 9B, the combination of mechanical and non-mechanical lysis has proven to be effective in lysis of Gram-positive bacteria. The highest signal was found using 3M NaOH for 5 minutes, 3M for 3.5 minutes and 2M for 5 minutes.

### Example 6. Testing Combination Lysis Methods on Eukaryotic Fungal Cells (Candida Albicans)

In this example, using the relevant materials and methodology described in Example 1, the effectiveness of different lysis methods was tested on different cell types, including Gram-negative cells, Gram-positive cells and eukaryotic fungal cells.

### Experimental Materials

1. The following bacterial samples were used:
   (a) 10 Gram-negative, including E. coli, P. mirabilis, K. pneumoniae, K. oxytoca, E. hormaechei, E. aerogenes, E. cloacae, P. aeruginosa, C. freundii, and S. marcescens
   (b) 9 Gram-positive organisms, including S. aureus, S. lugdunensis, E. faecalis, E. faecium, S. agalactiae, S. pneumoniae, S. viridans, and S. pyogenes
   (c) 1 yeast, C. albicans
2. All bacteria were grown in MH2+5% LAKED horse blood+1 ug/ml RnaseA
3. C. albicans was grown in RPMI overnight

### Experimental Methods

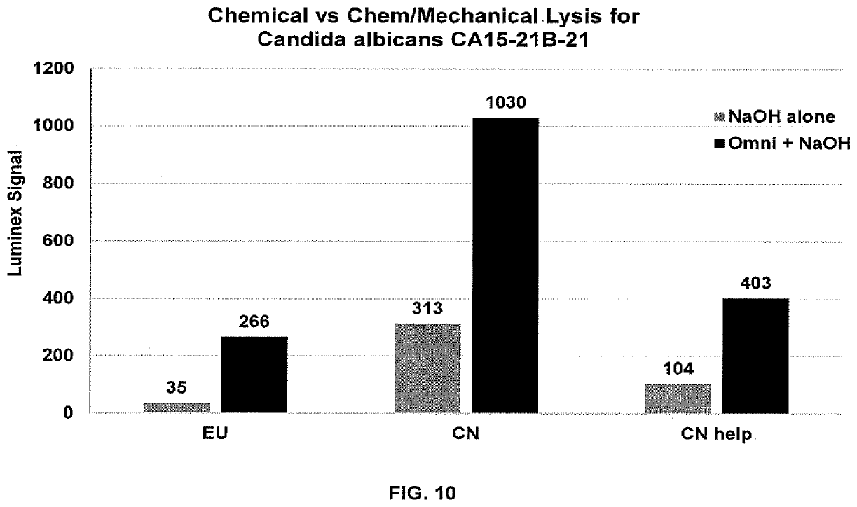

For Gram-negative cells, alkaline lysis alone was used. For Gram-positive cells, a combination of alkaline lysis with OmniLyse® mechanical lysis was used. For eukaryotic fungal cells both alkaline lysis alone and a combination of alkaline lysis with OmniLyse® mechanical lysis were tested and compared. When the combination was used, alkaline (chemical) lysis with 1M NaOH was performed for 5 minutes and Omnilyse® (mechanical) was performed for the first 2 minutes of the 5 minute alkaline (1M NaOH) lysis. Results for probe specificity following the lysis of each cell type are shown in FIG. 10.

### Results

As shown in FIG. 10, higher signals were obtained with the combination of chemical and mechanical lysis as detected with eumicrobial (EU) or candida (CN or CN-Help) probes.

### Example 7. Comparison of Buffers for Neutralizing Lysate

### Experimental Methods

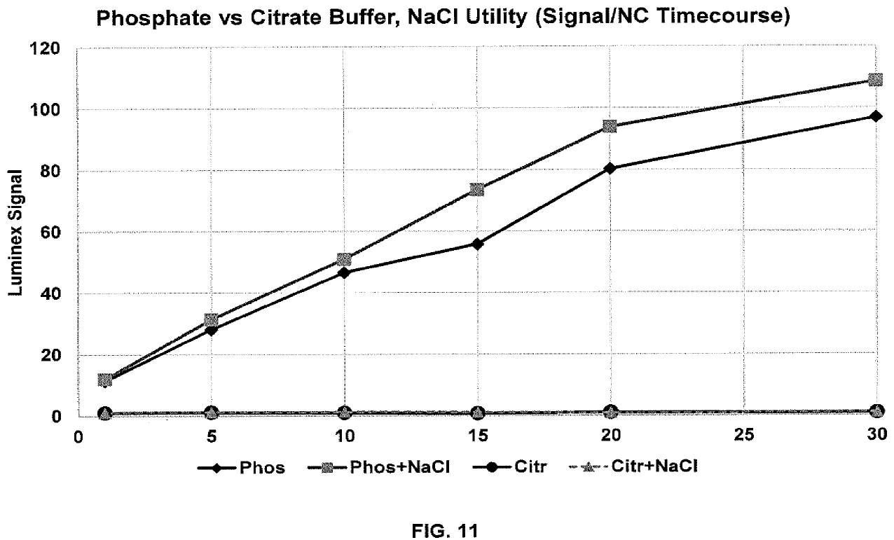

In this experiment, cell lysate samples were neutralized by contacting the samples with a buffer solution. During this experiment a series of different buffers were used, including: 1M Phosphate buffer (PB); 1M PB+1M NaCl; 1M Citrate buffer (CB); and 1M CB+1M NaCl and their ability to neutralize NaOH in the lysate was compared. See FIG. 11.

### Results

As shown in FIG. 11, when compared to an equal molarity strength of Citrate buffer, the phosphate buffer was much better at neutralizing the lysate.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A method for extracting a target nucleic acid from a cellular material in a sample, the method comprising the steps of:
(a) subjecting the sample to mechanical lysis within a lysis chamber to cause disruption of a cellular membrane in the cellular material;
(b) contacting the sample with an alkaline material within the lysis chamber to produce a lysate composition comprising the target nucleic acid; and
(c) recovering the lysate composition from the sample from the lysis chamber;
wherein steps (a) and (b) are carried out in the lysis chamber.

2. The method defined in claim 1, wherein the target nucleic acid is RNA.

3. The method defined in claim 1, wherein the target nucleic acid is ribosomal RNA (rRNA).

4. The method defined in claim 1, wherein Step (b) comprises contacting the cellular material in the sample with an alkaline solution and wherein the alkaline solution is a sodium hydroxide solution.

5. The method defined in claim 5, wherein the alkaline solution has a concentration of 10M or less.

6. The method defined in claim 1, wherein the cellular material comprises a microorganism.

7. The method defined in claim 1, wherein the cellular material comprises bacteria.

8. The method defined in claim 1, wherein the sample comprises a bodily fluid or an inoculant derived therefrom.

9. The method defined in claim 8, wherein the bodily fluid is selected from the group consisting of blood, urine, saliva, sweat, tears, mucus, breast milk, plasma, serum, synovial fluid, pleural fluid, lymph fluid, amniotic fluid, feces, cerebrospinal fluid and any mixture of two or more of these.

10. The method defined in claim 1, wherein Step (a) is conducted for a period of 10 minutes or less.

11. The method defined in claim 1, wherein the mechanical lysis is selected from the group consisting of French press, shaking, grinding, bead beating, centrifugation and any combination of two or more of these.

12. The method defined in claim 1, wherein the mechanical lysis comprises a combination of centrifugation and puck lysing.

13. The method defined in claim 1, wherein Steps (a) and (b) are carried out concurrently.

14. The method defined in claim 1, wherein Steps (a) and (b) are carried out sequentially.

15. The method defined in claim 1, wherein the method further comprises neutralizing the sample by contacting the sample with a buffer solution.

16. The method defined in claim 15 wherein the buffer solution has a pH in the range of from 6 to 7.

17. The method defined in claim 1, comprising the further step of detecting at least one nucleotide sequence in the cell lysate.

18. The method defined in claim 1, wherein Step (b) is carried out after commencement of disruption of the cellular membrane in Step (a).

19. The method defined in claim 1, wherein the mechanical lysis of step a) is performed while the alkaline material is present within the lysis chamber.
